(19) United States
(12) Patent Application Publication
(10) Pub. No.: US 2022/0117570 A1
(43) Pub. Date: Apr. 21, 2022

(54) SYSTEMS AND METHODS FOR CONTRAST FLOW MODELING WITH DEEP LEARNING

(71) Applicant: General Electric Company, Schenectady, NY (US)

(72) Inventors: Mark Vincent Profio, Elm Grove, WI (US); Eric Gros, Waukesha, WI (US); Christine Carol Hammond, Oconomowoc, WI (US); Darin Robert Okerlund, Muskego, WI (US)

(21) Appl. No.: 17/563,970

(22) Filed: Dec. 28, 2021

Related U.S. Application Data
(62) Division of application No. 15/881,526, filed on Jan. 26, 2018.

(51) Int. Cl. A61B 6/00; A61B 5/00; A61B 6/03; A61B 6/04

(52) U.S. Cl. CPC A61B 6/484; A61B 5/7264; A61B 6/40; A61B 6/42; A61B 6/54; A61B 6/0407; A61B 6/032

(57) ABSTRACT

Systems and methods are provided for contrast-enhanced diagnostic imaging. In one aspect, a system comprises an x-ray source that emits a beam of x-rays towards a subject to be imaged; a detector that receives the x-rays attenuated by the subject; a data acquisition system (DAS) operably connected to the detector; and a computing device operably connected to the DAS and configured with executable instructions in non-transitory memory that when executed cause the computing device to generate a first estimated time to perform a diagnostic scan of the subject based on demographic information and clinical information of the patient; and control the x-ray source and the detector to perform the diagnostic scan of the subject at the first estimated time responsive to a first confidence level of the first estimated time above a threshold.

### CROSS-REFERENCE TO RELATED APPLICATIONS

The present application is a divisional application of and claims priority to U.S. patent application Ser. No. 15/881,526, filed on Jan. 26, 2018, the entirety of which is incorporated herein by reference.

### BACKGROUND

Embodiments of the subject matter disclosed herein relate to non-invasive diagnostic imaging, and more particularly, to the optimization of contrast flow modeling with deep learning for diagnostic imaging.

Non-invasive imaging technologies allow images of the internal structures of a patient or object to be obtained without performing an invasive procedure on the patient or object. In particular, technologies such as computed tomography (CT) use various physical principles, such as the differential transmission of x-rays through the target volume, to acquire image data and to construct tomographic images (e.g., three-dimensional representations of the interior or the human body or of other imaged structures).

A contrast scan or exam, also referred to as an enhanced scan, is a scan in CT scan technologies where intravascular contrast media or contrast agents, such as iodine agents, barium sulfate, etc., are applied. The administration of a contrast media or bolus provides a short temporal window for optimally imaging the vasculature, lesions, and tumors. In order to image a region of interest (ROI) during this short temporal window, the contrast enhancement is sampled at regular intervals, such as every one or two seconds, to determine when to trigger the diagnostic scan. However, each sample acquired correspondingly increases the radiation dose administered to the patient.

### BRIEF DESCRIPTION

In an aspect, a method comprises estimating a time to perform a diagnostic scan of a patient based on demographics of the patient, and performing the diagnostic scan of the patient at the estimated time responsive to a confidence level of the estimated time above a threshold. In this way, a contrast-enhanced diagnostic scan may be performed without directly monitoring the contrast flow, thereby reducing radiation dose and contrast load while maintaining or improving image quality.

In another aspect, a method comprises estimating a time to perform a diagnostic scan of a patient based on demographics of the patient, and performing the diagnostic scan of the patient at the estimated time responsive to a confidence level of the estimated time above a threshold. In this way, a contrast-enhanced diagnostic scan may be performed without directly monitoring the contrast flow, thereby reducing radiation dose and contrast load while maintaining or improving image quality.

In yet another aspect, a system comprises an x-ray source that emits a beam of x-rays towards a subject to be imaged; a detector that receives the x-rays attenuated by the subject; a data acquisition system (DAS) operably connected to the detector; and a computing device operably connected to the DAS and configured with executable instructions in non-transitory memory that when executed cause the computing device to generate a first estimated time to perform a diagnostic scan of the subject based on demographic information and clinical information of the patient; and control the x-ray source and the detector to perform the diagnostic scan of the subject at the first estimated time responsive to a first confidence level of the first estimated time above a threshold.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present application will be better understood from reading the following description of non-limiting embodiments, with reference to the attached drawings, wherein below:

FIG. 1 shows a pictorial view of an imaging system according to an embodiment;

FIG. 2 shows a block schematic diagram of an exemplary imaging system according to an embodiment;

FIG. 3 shows a block diagram illustrating an example deep learning system for contrast flow modeling according to an embodiment;

FIG. 4 shows a high-level flow chart illustrating an example method for contrast flow modeling according to an embodiment; and

FIG. 5 shows a set of graphs illustrating an example timeline for a diagnostic scan according to an embodiment.

### DETAILED DESCRIPTION

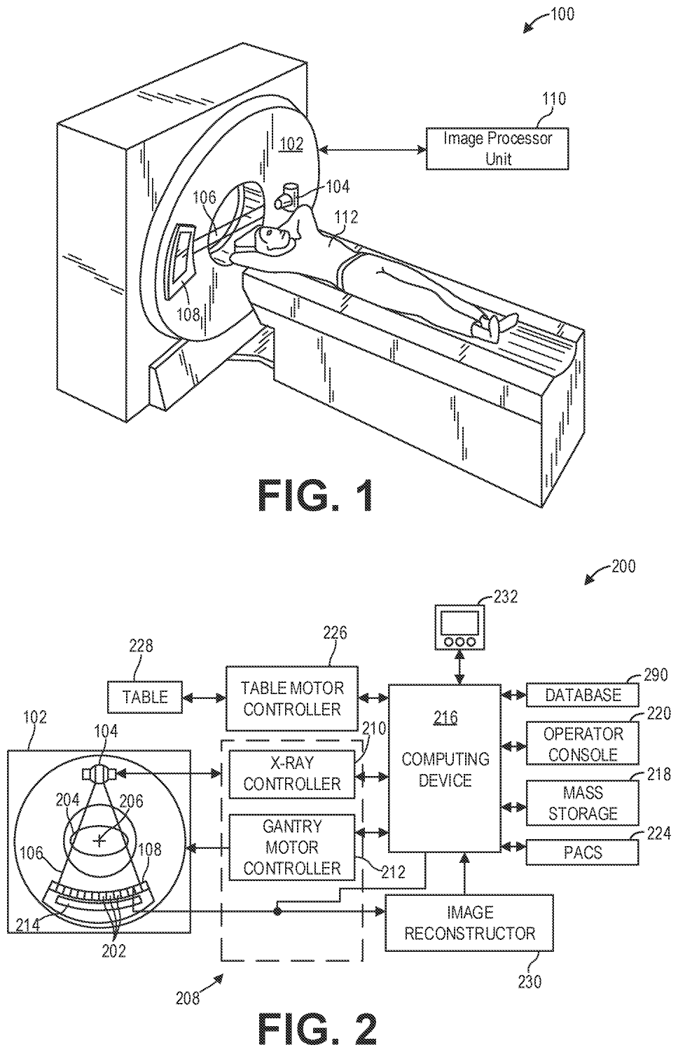

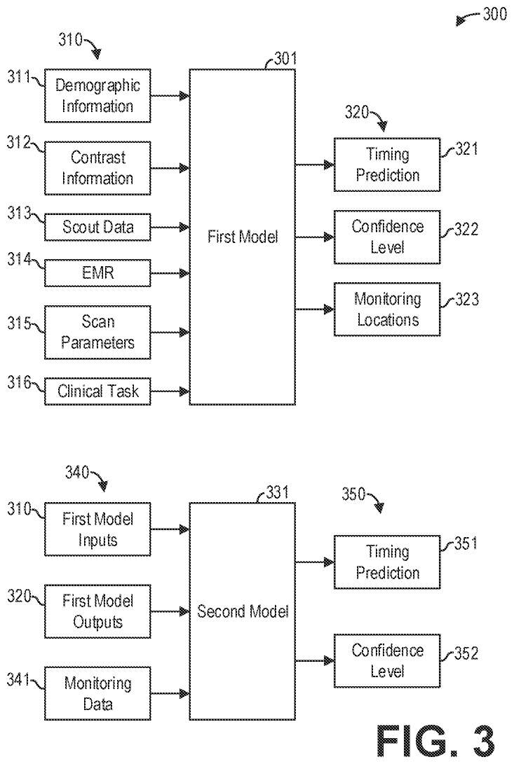

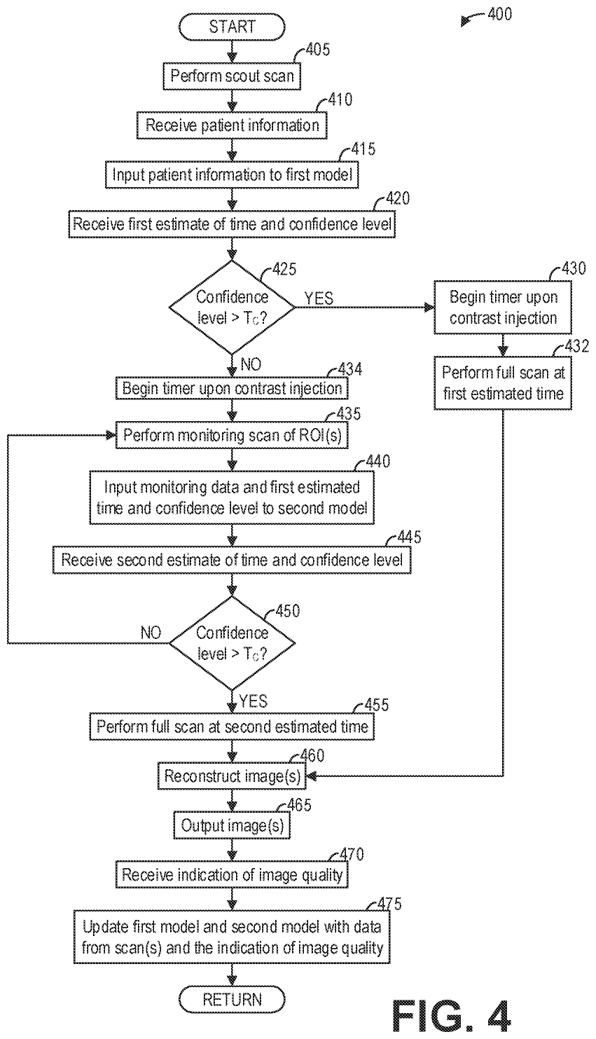

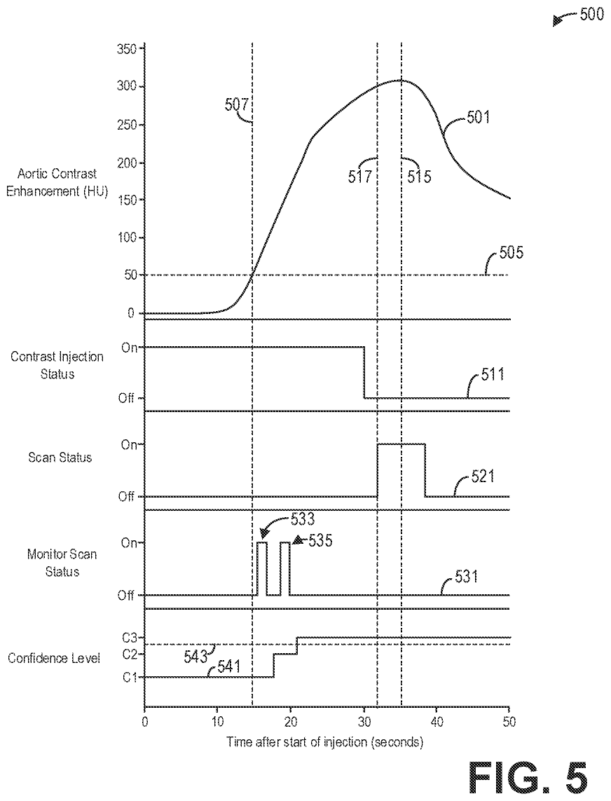

The following description relates to various embodiments of contrast-enhanced diagnostic imaging. In particular, systems and methods are provided for modeling contrast flow with deep learning for contrast-enhanced computed tomography (CT). An example of a CT imaging system that may be used to acquire images processed in accordance with the present techniques is provided in FIGS. 1 and 2. A deep learning system for modeling contrast flow, such as the deep learning system shown in FIG. 3, includes a first model for modeling the contrast flow based on a priori knowledge, as well as a second model for modeling the contrast flow based on the particular patient. A method for optimally performing a contrast-enhanced diagnostic scan, such as the method shown in FIG. 4, uses the first model and/or the second model to accurately predict a contrast enhancement event and trigger the diagnostic scan. FIG. 5 shows a timeline illustrating an example scan performed in accordance with the method of FIG. 4.

Contrast flow during CT exams has traditionally relied on anecdotal medical practice observation and expert interpretation to account for variables affecting flow rates and arrival times. Bolus tracking software requires additional monitoring scans and x-ray dose. Dual injection test bolus techniques require additional x-ray and contrast media dose. As described further herein, deep learning and other artificial intelligence techniques enable an accounting for patient demographics, clinical task/presentation, cardiac output, and relative tortuosity through vessels for patient to patient.

Though a CT system is described by way of example, it should be understood that the present techniques may also be useful when applied to images acquired using other imaging modalities, such as x-ray imaging systems, magnetic resonance imaging (MM) systems, positron emission tomography (PET) imaging systems, single-photon emission computed tomography (SPECT) imaging systems, ultrasound imaging systems, and combinations thereof (e.g., multi-modality imaging systems, such as PET/CT, PET/MR or SPECT/CT imaging systems). The present discussion of a CT imaging modality is provided merely as an example of one suitable imaging modality.

FIG. 1 illustrates an exemplary CT system 100 configured to allow fast and iterative image reconstruction. Particularly, the CT system 100 is configured to image a subject 112 such as a patient, an inanimate object, one or more manufactured parts, and/or foreign objects such as dental implants, stents, and/or contrast agents present within the body. In one embodiment, the CT system 100 includes a gantry 102, which in turn, may further include at least one x-ray radiation source 104 configured to project a beam of x-ray radiation 106 for use in imaging the subject 112. Specifically, the x-ray radiation source 104 is configured to project the x-rays 106 towards a detector array 108 positioned on the opposite side of the gantry 102. Although FIG. 1 depicts only a single x-ray radiation source 104, in certain embodiments, multiple x-ray radiation sources may be employed to project a plurality of x-rays 106 for acquiring projection data corresponding to the subject 112 at different energy levels.

In certain embodiments, the CT system 100 further includes an image processor unit 110 configured to reconstruct images of a target volume of the subject 112 using an iterative or analytic image reconstruction method. For example, the image processor unit 110 may use an analytic image reconstruction approach such as filtered backprojection (FBP) to reconstruct images of a target volume of the patient. As another example, the image processor unit 110 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the subject 112.

In some known CT imaging system configurations, a radiation source projects a fan-shaped beam which is collimated to lie within an X-Y plane of a Cartesian coordinate system and generally referred to as an “imaging plane.” The radiation beam passes through an object being imaged, such as the patient or subject 112. The beam, after being attenuated by the object, impinges upon an array of radiation detectors. The intensity of the attenuated radiation beam received at the detector array is dependent upon the attenuation of a radiation beam by the object. Each detector element of the array produces a separate electrical signal that is a measurement of the beam attenuation at the detector location. The attenuation measurements from all the detectors are acquired separately to produce a transmission profile.

In some CT systems, the radiation source and the detector array are rotated with a gantry within the imaging plane and around the object to be imaged such that an angle at which the radiation beam intersects the object constantly changes. A group of radiation attenuation measurements, i.e., projection data, from the detector array at one gantry angle is referred to as a “view.” A “scan” of the object includes a set of views made at different gantry angles, or view angles, during one revolution of the radiation source and detector. It is contemplated that the benefits of the methods described herein accrue to medical imaging modalities other than CT, so as used herein the term view is not limited to the use as described above with respect to projection data from one gantry angle. The term “view” is used to mean one data acquisition whenever there are multiple data acquisitions from different angles, whether from a CT, PET, or SPECT acquisition, and/or any other modality including modalities yet to be developed as well as combinations thereof in fused embodiments.

In an axial scan, the projection data is processed to reconstruct an image that corresponds to a two-dimensional slice taken through the object. One method for reconstructing an image from a set of projection data is referred to in the art as the filtered backprojection (FBP) technique. Transmission and emission tomography reconstruction techniques also include statistical iterative methods such as maximum likelihood expectation maximization (MLEM) and ordered-subsets expectation reconstruction techniques as well as iterative reconstruction techniques. This process converts the attenuation measurements from a scan into integers called “CT numbers” or “Hounsfield units,” which are used to control the brightness of a corresponding pixel on a display device.

To reduce the total scan time, a “helical” scan may be performed. To perform a helical scan, the patient is moved while the data for the prescribed number of slices is acquired. Such a system generates a single helix from a cone beam helical scan. The helix mapped out by the cone beam yields projection data from which images in each prescribed slice may be reconstructed.

As used herein, the phrase “reconstructing an image” is not intended to exclude embodiments of the present disclosure in which data representing an image is generated but a viewable image is not. Therefore, as used herein the term “image” broadly refers to both viewable images and data representing a viewable image. However, many embodiments generate (or are configured to generate) at least one viewable image.

FIG. 2 illustrates an exemplary imaging system 200 similar to the CT system 100 of FIG. 1. In accordance with aspects of the present disclosure, the imaging system 200 is configured to acquire three-dimensional (3D) scout scans and perform beam hardening corrections using data acquired during the 3D scout scan. In one embodiment, the imaging system 200 includes the detector array 108 (see FIG. 1). The detector array 108 further includes a plurality of detector elements 202 that together sense the x-ray beams 106 (see FIG. 1) that pass through a subject 204 such as a patient to acquire corresponding projection data. Accordingly, in one embodiment, the detector array 108 is fabricated in a multi-slice configuration including the plurality of rows of cells or detector elements 202. In such a configuration, one or more additional rows of the detector elements 202 are arranged in a parallel configuration for acquiring the projection data.

In certain embodiments, the imaging system 200 is configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where a projection angle relative to the subject 204 varies as a function of time, the mounted components may be configured to move along a general curve rather than along a segment of a circle.

As the x-ray radiation source 104 and the detector array 108 rotate, the detector array 108 collects data of the attenuated x-ray beams. The data collected by the detector array 108 undergoes pre-processing and calibration to condition the data to represent the line integrals of the attenuation coefficients of the scanned subject 204. The processed data are commonly called projections.

In dual or multi-energy imaging, two or more sets of projection data are typically obtained for the imaged object at different tube peak kilovoltage (kVp) levels, which change the peak and spectrum of energy of the incident photons comprising the emitted x-ray beams or, alternatively, at a single tube kVp level or spectrum with an energy resolving detector of the detector array 108.

The acquired sets of projection data may be used for basis material decomposition (BMD). During BMD, the measured projections are converted to a set of density line-integral projections. The density line-integral projections may be reconstructed to form a density map or image of each respective basis material, such as bone, soft tissue, and/or contrast agent maps. The density maps or images may be, in turn, associated to form a volume rendering of the basis material, for example, bone, soft tissue, and/or contrast agent, in the imaged volume.

Once reconstructed, the basis material image produced by the imaging system 200 reveals internal features of the subject 204, expressed in the densities of the two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, sizes and shapes of particular anatomies or organs, and other features that would be discernable in the image based upon the skill and knowledge of the individual practitioner.

In an embodiment, the imaging system 200 includes a control mechanism 208 to control movement of the components such as rotation of the gantry 102 and the operation of the x-ray radiation source 104. In certain embodiments, the control mechanism 208 further includes an x-ray controller 210 configured to provide power and timing signals to the x-ray radiation source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The data sampled and digitized by the DAS 214 is transmitted to a computer or computing device 216. In one example, the computing device 216 stores the data in a storage device such as mass storage 218. The mass storage 218, for example, may include a hard disk drive, a floppy disk drive, a compact disk-read/write (CD-R/W) drive, a Digital Versatile Disc (DVD) drive, a flash drive, and/or a solid-state storage drive.

Additionally, the computing device 216 provides commands and parameters to one or more of the DAS 214, the x-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) and/or a touchscreen to allow the operator to specify the commands and/or scanning parameters.

Although FIG. 2 illustrates only one operator console 220, more than one operator console may be coupled to the imaging system 200, for example, for inputting or outputting system parameters, requesting examinations, and/or viewing images. Further, in certain embodiments, the imaging system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks.

In one embodiment, for example, the imaging system 200 either includes or is coupled to a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control a table 228 which may comprise a motorized table. Particularly, the table motor controller 226 moves the table 228 for appropriately positioning the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized x-ray data to perform high-speed reconstruction. Although FIG. 2 illustrates the image reconstructor 230 as a separate entity, in certain embodiments, the image reconstructor 230 may form part of the computing device 216. Alternatively, the image reconstructor 230 may be absent from the imaging system 200 and instead the computing device 216 may perform one or more of the functions of the image reconstructor 230. Moreover, the image reconstructor 230 may be located locally or remotely, and may be operatively connected to the imaging system 200 using a wired or wireless network. Particularly, one exemplary embodiment may use computing resources in a “cloud” network cluster for the image reconstructor 230.

In one embodiment, the image reconstructor 230 stores the images reconstructed in the storage device or mass storage 218. Alternatively, the image reconstructor 230 transmits the reconstructed images to the computing device 216 for generating useful patient information for diagnosis and evaluation. In certain embodiments, the computing device 216 transmits the reconstructed images and/or the patient information to a display 232 communicatively coupled to the computing device 216 and/or the image reconstructor 230.

The various methods and processes described further herein may be stored as executable instructions in non-transitory memory on a computing device in imaging system 200. For example, image reconstructor 230 may include such executable instructions in non-transitory memory, and may apply the methods described herein to reconstruct an image from scanning data. In another embodiment, computing device 216 may include the instructions in non-transitory memory, and may apply the methods described herein, at least in part, to a reconstructed image after receiving the reconstructed image from image reconstructor 230. In yet another embodiment, the methods and processes described herein may be distributed across image reconstructor 230 and computing device 216.

In one embodiment, the display 232 allows the operator to evaluate the imaged anatomy. The display 232 may also allow the operator to select a volume of interest (VOI) and/or request patient information, for example, via a graphical user interface (GUI) for a subsequent scan or processing.

Computing device 216 may be configured with executable instructions in non-transitory memory that when executed cause the computing device 216 to generate estimates of contrast flow timing events. For example, the computing device 216 may predict when peak contrast enhancement occurs for a particular patient. To that end, a deep learning system (not shown) may be implemented on computing device 216. An example deep learning system is described further herein with regard to FIG. 3.

To train the deep learning system as well as generate predictions with the deep learning system, the computing device 216 may retrieve data from one or more databases. Thus, in one embodiment, the computing device 216 is communicatively coupled to one or more databases 290, for example via a wired or wireless network (not shown). The one or more databases 290 may include, but are not limited to, a radiology information system (RIS) database, a hospital information system (HIS) database, an electronic medical record (EMR) database, and so on.

FIG. 3 shows a block diagram illustrating a deep learning system 300 for contrast flow modeling according to an embodiment. In particular, FIG. 3 illustrates the inputs and outputs of a first deep learning model 301, hereinafter referred to as first model 301, and a second deep learning model 331, hereinafter referred to as second model 331. As mentioned hereinabove, the deep learning system 300 may be implemented with a computing system such as computing device 216. In general, the deep learning system 300 is configured to predict when a contrast flow event, such as peak or maximum contrast enhancement, occurs.

The first model 301 receives a plurality of inputs 310 and generates a plurality of outputs 320 responsive to the plurality of inputs 310. The plurality of inputs 310 may include, as exemplary and non-limiting examples, demographic information 311 corresponding to the patient to be scanned, contrast information 312 regarding the contrast injection for the patient, scout data 313 acquired during a scout scan of the patient, and an EMR 314 for the patient. The plurality of outputs 320 may include, as exemplary and non-limiting examples, a timing prediction 321 and a confidence level 322 for the timing prediction 321. Additionally, in some examples, the plurality of outputs 320 further includes one or more monitoring locations 323.

The first model 301 is trained with a training dataset containing information from a plurality of contrast-enhanced scans. More specifically, each entry of the training dataset corresponds to a single contrast-enhanced scan, and each entry includes, as exemplary and non-limiting examples, demographic information for a patient, EMR for the patient, the clinical task for the scan, contrast injection settings, the scan and reconstruction settings, diagnostic image sets from the scan, and contrast enhancement over time. In some examples, each entry may further include image quality metrics for the diagnostic images. For example, the image quality metrics may include quantitative metrics which quantify image noise and/or image texture, as well as qualitative metrics which indicate whether the image was useful for diagnosis. In this way, the first model 301 may learn relationships between image quality and the demographics, clinical task, contrast injection settings, scan settings, reconstruction settings, and the contrast enhancement over time.

To that end, the first model 301 may comprise a supervised or a partially supervised deep machine learning algorithm or algorithms. For example, the first model 301 may comprise a combination of random forest(s) and support vector machine(s). As another example, the first model 301 may comprise a combination of recurrent neural networks and convolutional neural networks. In some examples, the first model 301 may comprise a long short-term memory network (LSTM). Additionally or alternatively, the first model 301 may comprise an unsupervised deep learning algorithm or algorithms. For example, the first model 301 may include a neural network that undergoes unsupervised training to identify monitoring locations 323 based on the scout data 313 and other inputs of the plurality of inputs 310. The monitoring locations 323 comprise one or more regions of interest (ROIs) that may be imaged to evaluate the contrast enhancement at a given time. It should be appreciated that the first model 301 may include a neural network that instead undergoes supervised training to identify the monitoring locations 323.

Similar to the first model 301, the second model 331 of the deep learning system 300 receives a plurality of inputs 340 and generates a plurality of outputs 350 responsive to the plurality of inputs 340. The plurality of inputs 340 may include, as exemplary and non-limiting examples, the plurality of inputs 310, also referred to herein as the first model inputs 310. The plurality of inputs 340 further includes the plurality of outputs 320, also referred to herein as the first model outputs 320. The plurality of inputs 340 may further include monitoring data 341 acquired during one or more monitoring scans. The monitoring scans comprise short, low-dose scans of the one or more monitoring locations 323.

The plurality of outputs 350 includes a timing prediction 351 and a confidence level 352 for the timing prediction 351. As second model 331 estimates the timing prediction 351 based on the monitoring data 341, the timing prediction 351 is likely more accurate than the timing prediction 321, and thus the confidence level 352 is likely higher than the confidence level 322, because the monitoring data 341 provides data regarding how contrast is flowing through the patient's body. As mentioned above, multiple monitoring locations 323 may be scanned to acquire the monitoring data 341. In this way, a more dynamic view of how contrast is flowing through the patient's body may be captured, as the contrast enhancement in each monitoring location 323 may be different. As an illustrative and non-limiting example, the monitoring locations 323 may include the descending aorta, the ascending aorta, the left ventricle, the right ventricle, the left atrium, and the right atrium. One monitor scan that acquires projection data simultaneously in each monitoring location 323 or ROI therefore provides six different data points illustrating how contrast is flowing through the heart.

In some instances, the first model 301 may be trained on a sufficient amount of data such that the confidence level 322 of the timing prediction 321 output by the first model 301 is greater than a threshold confidence level. In such instances, monitoring locations 323 may not be output, monitoring scans of the monitoring locations 323 may not be performed, and the second model 331 may not receive any inputs or generate any outputs.

To model contrast flow, a loss function of the first model 301 and/or the second model 331 may be defined in terms of exceeding a level of HU enhancement for a defined contrast-enhanced study. Baseline image quality metrics may include image noise, image texture, x-ray dose, scan time, and contrast load. The models may be differentiated by patient information, clinical task, and scan time. The scan time may be further refined to include coverage distance, rotational speed, helical pitch and collimation or axial collimation, and number of slabs. To train the first model 301 and the second model 331, a curated training database may be assembled that includes EMR patient information, similar clinical tasks, scan and reconstruction settings, and diagnostic image sets including any patient-specific prior studies. The diagnostic image sets are filtered and binned against the baseline image quality metrics. A loss function is determined for each image set based on the difference from a desired HU enhancement level. The qualifying exam results may be differentiated by EMR patient information, clinical task, and the scan and reconstruction settings. The deep learning models define a good diagnostic image outcome and finds similar image sets throughout the curated training database. Best outcome image datasets provide insight into scan and reconstruction settings that are most often or most likely to result in exceeding the desired HU enhancement level. The deep learning models may further recommend scan and reconstruction settings as well as prescribe contrast parameters for a particular patient.

The demographic information 311 may include information such as weight, height, age, gender, and so on. In some examples, the demographic information 311 may further include information such as cardiac output, venous access, breath-holding, disease state, renal function, and so on. The clinical task 316 may include information such as target organs for imaging. The scan parameters 315 may include information such as scan duration, scan delay, multi-phase scan, scan direction, ECG-gating, and so on. The contrast information 312 may include information such as iodine mass (e.g., concentration, volume), rate, saline flush, injection duration (e.g., volume, rate), viscosity, injection pattern (e.g., uniphase, biphase, exponentially-decay), and so on.

FIG. 4 shows a high-level flow chart illustrating an example method 400 for contrast flow modeling according to an embodiment. In particular, method 400 relates to using a first deep learning model and/or a second deep learning model to accurately predict when peak contrast enhancement will occur and performing a diagnostic scan based on the prediction. Method 400 is described with regard to the systems and components of FIGS. 1-3, though it should be appreciated that the method may be implemented with other systems and components without departing from the scope of the present disclosure. Method 400 may be implemented as executable instructions in non-transitory memory of a computing device, such as computing device 216.

Method 400 begins at 405. At 405, method 400 performs a scout scan of a subject, such as a patient. The scout scan may comprise an axial or a helical low-dose scan of the patient. The scout data acquired during the scout scan may be used to perform patient anatomy localization and/or automatic exposure control for the full diagnostic scan, as non-limiting examples.

At 410, method 400 receives patient information. The patient information may include, as exemplary and non-limiting examples, demographic information regarding the patient, an EMR for the patient, scout data acquired during the scout scan of the patient at 405, contrast information regarding the contrast prescription for the patient, and so on.

Continuing at 415, method 400 inputs the patient information to a first model, such as the first model 301, that predicts a desired time to perform the full diagnostic scan. In some examples, the desired time to perform the full diagnostic scan corresponds to the time of maximum contrast enhancement. However, it should be appreciated that the desired time may correspond to a threshold other than the maximum contrast enhancement, and so the first model may predict the time that corresponds to the other threshold. The first model further determines a confidence level regarding the predicted time. At 420, method 400 receives a first estimate of time and confidence level from the first model. In some examples, the method 400 further receives an indication of one or more monitoring locations or ROIs for targeted monitoring scans from the first model.

At 425, method 400 determines if the confidence level is above a threshold confidence level T_{C}. The threshold confidence level T_{C} may be selected to ensure that the full diagnostic scan performed based on the estimated time acquires sufficiently reliable data for diagnosis. As an illustrative and non-limiting example, the threshold confidence level T_{C} may comprise 99%. For example, if the confidence level is at least 99%, then there is at least a 99% chance that the selected event (e.g., maximum contrast enhancement) occurs at the first estimate of time. It should be appreciated that in some examples, the threshold confidence level T_{C} is lower than 99%. For example, the threshold confidence level T_{C} may comprise 95%.

If the confidence level is above the threshold confidence level T_{C} (“YES”), then method 400 proceeds to 430. At 430, method 400 begins a timer upon contrast injection. That is, the timer begins upon injection of contrast into the patient. At 432, method 400 performs the full diagnostic scan based on the first estimated time as counted by the timer. In some examples, the first estimated time may correspond to the time when the diagnostic scan should begin. However, if the first estimated time corresponds to a specific contrast enhancement event that should be imaged (e.g., maximum contrast enhancement), then in some examples the full diagnostic scan may be triggered prior to the first estimated time. In such examples, the timing for triggering the full diagnostic scan may be determined based on the first estimated time. For example, if it is desired to image the patient during maximum contrast enhancement, then the duration of the full diagnostic scan may be centered on the first estimated time. In this example, the time to trigger the full diagnostic scan may comprise the first estimated time minus half of the duration. After performing the full diagnostic scan, method 400 continues to 460.

However, referring again to 425, if the confidence level is below the threshold confidence level T_{C} (“NO”), method 400 proceeds to 434. At 434, method 400 begins the timer upon the contrast injection. Continuing at 435, method 400 performs a monitoring scan of one or more ROIs. The monitoring scan comprises a short low-dose scan of the one or more ROIs. In some examples, the one or more ROIs comprise monitoring locations automatically determined by the first model, as described hereinabove. In other examples, the one or more ROIs are manually selected by a user.

After performing the monitoring scan of the one or more ROIs, method 400 continues to 440. At 440, method 400 inputs the monitoring data, the first estimated time, and the first confidence level to a second model, such as the second model 331 described hereinabove, configured to predict a desired time to perform the full diagnostic scan. The monitoring data comprises the projection data acquired during the monitoring scan at 435. Thus, rather than estimating the time to perform the full diagnostic scan based solely on prior scans performed on other patients, the second model estimates the time based on such prior scans as well as a current contrast-enhanced scan of the patient. Thus, at 445, method 400 receives a second estimate of time and a second confidence level for the second estimate of time generated by the second model based on the inputs to the second model.

At 450, method 400 determines if the second confidence level is above the threshold contrast level T_{C}. The threshold contrast level T_{C} comprises the same threshold contrast level T_{C} used at 425.

If the second confidence level is below the confidence level threshold T_{C} (“NO”), method 400 returns to 435. Method 400 thus performs a second monitoring scan of the one or more ROIs at 435. Method 400 then inputs the monitoring data acquired during the second monitoring scan, the second estimate of time, and the second confidence level to the second model at 440. Method 400 then receives a third estimate of time and a third confidence level regarding the third estimate of time from the second model at 445. Method 400 may thus perform monitoring scans of the one or more ROIs and update an estimate of the time until the confidence level output by the second model is greater than or equal to the threshold confidence level T_{C} at 450. For simplicity, the estimated time and confidence level output by the second model at 445 are referred to hereafter as the second estimated time and the second confidence level, regardless of how many iterations of the loop between 450 and 435 method 400 performs.

Referring again to 450, once the second confidence level (or the most recent confidence level output by the second model at 445) is above the threshold confidence level (“YES”), method 400 proceeds to 455. At 455, method 400 performs a full diagnostic scan based on the second estimated time (or the most recent estimated time output by the second model at 445). In particular, method 400 performs the full diagnostic scan based on the second estimated time as counted by the timer initiated at 434. As discussed hereinabove, in some examples, the second estimated time may correspond to the time when the diagnostic scan should begin. However, if the second estimated time corresponds to a specific contrast enhancement event that should be imaged (e.g., maximum contrast enhancement), then in some examples the full diagnostic scan may be triggered prior to the second estimated time. In such examples, the timing for triggering the full diagnostic scan may be determined based on the second estimated time. For example, if it is desired to image the patient during maximum contrast enhancement, then the duration of the full diagnostic scan may be centered on the second estimated time. In this example, the time to trigger the full diagnostic scan may comprise the second estimated time minus half of the duration. After performing the full diagnostic scan, method 400 continues to 460.

At 460, method 400 reconstructs one or more images from the projection data acquired during the full diagnostic scan performed at 455 or 432. Method 400 may reconstruct the one or more images by applying an analytic or iterative image reconstruction algorithm to the acquired projection data. After reconstructing the one or more images, method 400 outputs the one or more images at 465. For example, method 400 may output the one or more images to a display device, such as display 232. Additionally or alternatively, method 400 may output the one or more images to a storage device such as mass storage 218 and/or a picture archiving and communication system such as PACS 224.

At 470, method 400 receives an indication of image quality. Method 400 may receive the indication of image quality, for example, via an operator console such as operator console 220. The indication of image quality indicates whether the one or more images output at 465 were successful or useful for diagnosis. In some examples, the indication of image quality may comprise a simple classification; for example, the indication of image quality may comprise “Yes” if the image quality is sufficient or “No” if the image quality is insufficient for diagnosis. In other examples, the indication of image quality may use a scale to indicate the image quality. As an illustrative and non-limiting example, a user may select a number ranging from 1 to 10, wherein 1 corresponds to an unusable image and 10 corresponds to an excellent and high quality image (as subjectively determined by the user or evaluator of the image). In other examples, a combination of image quality metrics may be used.

At 475, method 400 updates the first model and the second model with data from the scan(s) and the indication of image quality. In this way, the execution of method 400 provides a new data point for training the first and second models. By including the indication of image quality as subjectively determined by the user, the first and second models may adapt to provide better timing predictions that yield higher quality images in subsequent executions of the method. Method 400 then ends.

FIG. 5 shows a set of graphs illustrating an example timeline 500 for a diagnostic scan according to an embodiment. In particular, the timeline 500 illustrates how a diagnostic scan may be performed in accordance with method 400 described hereinabove. The timeline 500 depicts an aortic contrast enhancement 501, a contrast injection status 511, a scan status 521, a monitor scan status 531, and a confidence level 541 over time. The aortic contrast enhancement 501 illustrates contrast enhancement measured in an aorta in terms of Hounsfield units (HU), the contrast injection status 511 illustrates whether contrast is being injected (“On”) or not (“Off”), the scan status 521 illustrates whether the full diagnostic scan is being performed (“On”) or not (“Off”), the monitor scan status 531 illustrates whether a monitor scan is being performed (“On”) or not (“Off”), and the confidence level 541 illustrates the confidence level for the estimated timing output by the first and/or second models.

The contrast injection begins when the time equals zero seconds, as indicated by the contrast injection status 511. As indicated by the confidence level 541, initially the first model 331 outputs a first confidence level C1 that a first estimated time for the peak or maximum contrast enhancement is accurate. Since the first confidence level C1 is below the threshold confidence level 543, a monitoring scan is performed to update the estimated time.

Initially, there may be no contrast enhancement in the ROI (e.g., the aorta), as indicated by the aortic contrast enhancement 501. Thus, a monitoring scan may not be performed until a non-zero contrast enhancement is expected, as depicted by monitor scan status 531. To that end, a monitoring scan may be performed at a contrast arrival time 507 that corresponds to the aortic contrast enhancement 501 reaching a contrast bolus-tracking threshold 505. The contrast arrival time 507 may be predetermined based on statistical analysis of previous scans, as a non-limiting example. In some examples, the first model may output an estimated contrast arrival time in addition to the estimated time for the contrast event (e.g., peak contrast enhancement). The monitoring scan may be performed at the contrast arrival time 507. However, to ensure that the contrast enhancement 501 is above the contrast bolus-tracking threshold 505, the monitor scan 533 may be performed a predetermined duration after the contrast arrival time 507, as depicted by monitor scan status 531. The monitor scan 533 comprises a short-duration, low-dose scan of one or more ROIs such as the aorta.

The monitor data acquired during the monitor scan 533 is input to the second model 331, for example, to generate a second estimate of time and a second confidence level. As indicated by confidence level 541, the second confidence level C2 output by the second model is greater than the first confidence level C1 output by the first model, but less than the confidence level threshold 543. Therefore, a second monitor scan 535 is performed, as indicated by monitor scan status 531. The monitor data acquired during the second monitor scan 535 is input to the second model 331 to generate a third estimate of time and a third confidence level. As indicated by confidence level 541, the third confidence level C3 is greater than the confidence level threshold 543. No additional monitor scans may be performed once the confidence level 541 is above the confidence level threshold 543. Further, the monitor scans 533 and 535 may be performed while contrast is still being injected, as depicted by contrast injection status 511 and monitor scan status 531.

The aortic contrast enhancement 501 depicts peak contrast enhancement occurring at time 515, though the full diagnostic scan begins at time 517 prior to the time 515 as indicated by scan status 521. In some examples, the first and second models predict and output the time 515 at which peak contrast enhancement occurs, and the scan delay or time 517 is determined based on the time 515. In other examples, the first and second models output the scan delay or time 517 at which the full diagnostic scan is triggered.

Thus, as an illustrative example, the first model 301 may initially predict that the peak contrast enhancement occurs at 38 seconds plus or minus 5 seconds, where 38 seconds is the first estimate of time and the plus or minus 5 seconds corresponds to the first confidence level C1. Based on the monitor data acquired during monitor scan 533, the second model 331 then predicts that peak contrast enhancement occurs at 36 seconds plus or minus 2 seconds, where 36 seconds comprises the second estimate of time and the plus or minus 2 seconds corresponds to the second confidence level C2. Based on the monitor data acquired during monitor scan 535, the second model 331 predicts that peak contrast enhancement occurs at 35 seconds plus or minus 1 second, where 35 seconds is the third estimate of time and the plus or minus 1 second corresponds to the third confidence level C3. The scan delay or time 517 is then determined so that the duration of the full diagnostic scan is centered on 35 seconds after the start of injection.

Thus, systems and methods are provided for modeling contrast flow by leveraging previously-acquired data and deep learning techniques. If the models are trained with a large number of data points for a particular demographic and clinical task, for example, then the first model 301 may generate an estimated time with a high enough confidence level that no monitoring scans are necessary. Thus, in some cases, the full diagnostic scan may be accurately triggered without performing any monitoring scans. However, in some instances the first model 301 may not be trained on enough data for a particular demographic and/or clinical task, and so the first confidence level may be lower. The second model 331 thus refines the estimated time by utilizing data specific to the patient, namely the projection data acquired during the monitor scan(s). Over time, as more data regarding contrast enhancement timing and image quality is collected for different demographics and clinical tasks, the accuracy of the first model and the second model improves.

The systems and methods described herein provide user guidance and decision support for optimized scan and reconstruction settings to help ensure a low-dose, clinically diagnostic, and appropriately contrast-enhanced image dataset. This capability increases repeatability and robustness, thereby lowering re-scan rates, read errors, and missed incidentals. A suite of clinical tasks for different population demographics can be analyzed to target specific imaging diagnosis scenarios, covering the desired breadth and depth at a given institution. Patient care is improved, with lower x-ray dose and contrast dose, as well as lower costs given the minimization or elimination of re-scans.

A technical effect of the disclosure is the optimal timing of a contrast-enhanced diagnostic scan without monitoring contrast uptake. Another technical effect of the disclosure is the optimal timing of a contrast-enhanced scan with minimal sampling of the contrast uptake. Yet another technical effect of the disclosure is the execution of a contrast-enhanced diagnostic scan with reduced radiation dose, reduced contrast dose, and improved image quality. Another technical effect of the disclosure is the improvement over time of contrast-enhanced diagnostic imaging for patients with similar demographics.

In one embodiment, a method comprises estimating a time to perform a diagnostic scan of a patient based on demographics of the patient, and performing the diagnostic scan of the patient at the estimated time responsive to a confidence level of the estimated time above a threshold.

In a first example of the method, the method further comprises performing a monitor scan of one or more regions of interest (ROIs) of the patient responsive to the confidence level below the threshold, estimating a second time to perform the diagnostic scan based on the demographics of the patient and the monitor scan, and performing the diagnostic scan at the second estimated time responsive to a confidence level of the second estimated time above the threshold. In a second example of the method optionally including the first example, the monitor scan comprises a short-duration, low-dose scan relative to the diagnostic scan. In a third example of the method optionally including one or more of the first and second examples, the method further comprises estimating the time with a first deep learning model trained on previously-acquired scans of other patients, and estimating the second time with a second deep learning model that evaluates data acquired during the monitor scan. In a fourth example of the method optionally including one or more of the first through third examples, the first deep learning model comprises one or more of a recurrent neural network, a convolutional neural network, a random forest, and a support vector machine, and wherein the second deep learning model comprises one or more of a recurrent neural network and a convolutional neural network. In a fifth example of the method optionally including one or more of the first through fourth examples, the method further comprises reconstructing an image from data acquired during the diagnostic scan, receiving an indication of image quality for the image, and updating one or more of the first deep learning model and the second deep learning model based on the indication of image quality. In a sixth example of the method optionally including one or more of the first through fifth examples, the method further comprises automatically determining, with the first deep learning model, the one or more ROIs. In a seventh example of the method optionally including one or more of the first through sixth examples, the time is further estimated based on one or more of contrast injection parameters for the patient, an electronic medical record of the patient, scan parameters, and a clinical task. In an eighth example of the method optionally including one or more of the first through the seventh examples, the time corresponds to a contrast-enhancement event. In a ninth example of the method optionally including one or more of the first through eighth examples, the contrast-enhancement event comprises a peak contrast enhancement.

In another embodiment, a method comprises generating a first estimated time for triggering a diagnostic scan of a patient and a first confidence level for the first estimated time based on demographic information and clinical information relating to the patient, determining that the first confidence level is below a threshold, performing a scan of an ROI at a low dose for a short duration to measure a contrast enhancement in the ROI, generating a second estimated time for triggering the diagnostic scan and a second confidence level for the second estimated time based on data acquired during the scan, and performing the diagnostic scan at the second estimated time responsive to the second confidence level above the threshold.

In a first example of the method, generating the first estimated time comprises inputting the demographic information and the clinical information relating to the patient to a first deep learning model, and receiving the first estimated time and the first confidence level from the first deep learning model. In a second example of the method optionally including the first example, generating the second estimated time and the second confidence level comprises inputting the demographic information, the clinical information, and the data acquired during the scan to a second deep learning model, and receiving the second estimated time and the second confidence level from the second deep learning model. In a third example of the method optionally including one or more of the first and second examples, the method further comprises automatically determining the ROI with the first deep learning model. In a fourth example of the method optionally including one or more of the first through third examples, the first estimated time and the second estimated time comprise estimates of maximum contrast enhancement.

In yet another embodiment, a system comprises an x-ray source that emits a beam of x-rays towards a subject to be imaged, a detector that receives the x-rays attenuated by the subject, a data acquisition system (DAS) operably connected to the detector, and a computing device operably connected to the DAS and configured with executable instructions in non-transitory memory that when executed cause the computing device to generate a first estimated time to perform a diagnostic scan of the subject based on demographic information and clinical information of the patient, and control the x-ray source and the detector to perform the diagnostic scan of the subject at the first estimated time responsive to a first confidence level of the first estimated time above a threshold.

In a first example of the system, the computing device is further configured with executable instructions in non-transitory memory that when executed cause the computing device to control the x-ray source and the detector to perform a monitoring scan of an ROI of the subject responsive to the first confidence level below the threshold, the monitoring scan comprising a low-dose, short-duration scan relative to the diagnostic scan, generate a second estimated time to perform the diagnostic scan of the subject based on projection data acquired during the monitoring scan, and control the x-ray source and the detector to perform the diagnostic scan of the subject at the second estimated time responsive to a second confidence level of the second estimated time above the threshold. In a second example of the system optionally including the first example, the computing device is configured with a first deep learning model and a second deep learning model, wherein the first deep learning model generates the first estimated time and the second deep learning model generates the second estimated time. In a third example of the system optionally including one or more of the first and second examples, the computing device is further configured with executable instructions in non-transitory memory that when executed cause the computing device to reconstruct an image from data acquired during the diagnostic scan, receive, via an operator console communicatively coupled to the computing device, an indication of image quality for the image, and update one or more of the first deep learning model and the second deep learning model based on the indication of image quality. In a fourth example of the system optionally including one or more of the first through third examples, the first estimated time comprises a timing prediction of peak contrast enhancement in an ROI of the subject.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A system, comprising:
an x-ray source that emits a beam of x-rays towards a subject to be imaged;
a detector that receives the x-rays attenuated by the subject;
a data acquisition system (DAS) operably connected to the detector; and
a computing device operably connected to the DAS and configured with executable instructions in non-transitory memory that when executed cause the computing device to:
generate a first estimated time to perform a diagnostic scan of the subject based on demographic information and clinical information of the patient; and
control the x-ray source and the detector to perform the diagnostic scan of the subject at the first estimated time responsive to a first confidence level of the first estimated time above a threshold.

2. The system of claim 1, wherein the computing device is further configured with executable instructions in non-transitory memory that when executed cause the computing device to:
control the x-ray source and the detector to perform a monitoring scan of a region of interest (ROI) of the subject responsive to the first confidence level below the threshold, the monitoring scan comprising a low-dose, short-duration scan relative to the diagnostic scan;
generate a second estimated time to perform the diagnostic scan of the subject based on projection data acquired during the monitoring scan; and
control the x-ray source and the detector to perform the diagnostic scan of the subject at the second estimated time responsive to a second confidence level of the second estimated time above the threshold.

3. The system of claim 2, wherein the computing device is configured with a first deep learning model and a second deep learning model, wherein the first deep learning model generates the first estimated time and the second deep learning model generates the second estimated time.

4. The system of claim 3, wherein the computing device is further configured with executable instructions in non-transitory memory that when executed cause the computing device to:
reconstruct an image from data acquired during the diagnostic scan;
receive, via an operator console communicatively coupled to the computing device, an indication of image quality for the image; and
update one or more of the first deep learning model and the second deep learning model based on the indication of image quality.

5. The system of claim 1, wherein the first estimated time comprises a timing prediction of peak contrast enhancement in a region of interest (ROI) of the subject.
